(19) United States
(12) Patent Application Publication
(10) Pub. No.: US 2017/0076628 A1
(43) Pub. Date: Mar. 16, 2017

(54) Talk In A Box

(71) Applicant: Christine Marineau, Coram, NY (US)

(72) Inventor: Christine Marineau, Coram, NY (US)

(21) Appl. No.: 14/854,364

(22) Filed: Sep. 15, 2015

(51) Int. Cl. G09B 19/00; A61F 13/472; A61F 6/04; G09B 5/06

(52) U.S. Cl. CPC G09B 19/00; G09B 19/0076; G09B 5/065; A61F 13/472; A61F 6/04

(57) ABSTRACT

The current invention disclosure discloses a specially designed educational kit to help young adolescents learn about their sexuality and sex in a more effective manner. The kit is comprised of plurality of components, such as a booklet, a DVD, a sanitary napkin, a condom, details about personal hygiene, and other accessories.

### CROSS REFERENCE TO RELATED APPLICATION

Not Applicable

### FEDERALLY SPONSORED RESEARCH AND DEVELOPMENT Not Applicable

### MICROFICHE APPENDIX

Not Applicable

### BACKGROUND OF THE INVENTION

(1) FIELD OF INVENTION

The current invention disclosure pertains to an educational kit that is comprised of a plurality of components arranged in requisite order to facilitate an understanding of sex education.

(2) BACKGROUND OF INVENTION

It has been observed that early teens are in a transitional stage of physical and psychological human development that generally occurs during the period from puberty to legal adulthood (age of majority). Adolescence is not only a biological change, but is also a physical transition marked by the onset of puberty and the termination of physical growth; cognitively, as changes in the ability to think abstractly and multi-dimensionally; or socially, as a period of preparation for adult roles.

Major pubertal and biological changes include changes to the sex organs, height, weight, and muscle mass, as well as major changes in brain structure and organization. Cognitive advances encompass both increases in knowledge and in the ability to think abstractly and to reason more effectively.

The study of adolescent development often involves interdisciplinary collaborations. For example, researchers in neuroscience or bio-behavioral health might focus on pubertal changes in brain structure and its effects on cognition or social relations. Sociologists interested in adolescence might focus on the acquisition of social roles (e.g., worker or romantic partner) and how this varies across cultures or social conditions. Developmental psychologists might focus on changes in relations with parents and peers as a function of school structure and pubertal status.

Numerous attempts have been made in the past to create customized educational kits, educational DVD's, etc., as disclosed in prior arts, such as U.S. 4969821 A, CA 2159840 A1, and CA 2605993 A1, in order to educate children about sex without any interference from parents.

It has been observed that parents are generally apprehensive to address these changes, and seldom discuss their impacts. This is widely due to the social conditioning around them. Therefore, to overcome this communication barrier, there exist a need to effectively break this silence between the parents and the teenager.

In this regard, the present invention substantially fulfills this need.

### BRIEF SUMMARY OF THE INVENTION

In view of the foregoing disadvantages inherent in the known types of educational kits and techniques now present in the prior art, the present invention provides an improved and complete kit; wherein the same can be utilized to educate the child without parental assistance. As such, the general purpose of the present invention, which will be described subsequently in greater detail, is to provide a complete kit to facilitate an understanding about sex education to young teenagers. It is further stated that this education kit has all of the advantages of the prior art educational tools and techniques, and none of the disadvantages.

The following summary is provided to facilitate an understanding of some of the innovative features unique to the disclosed embodiment, and is not intended to be a full description. A full appreciation of the various aspects of the embodiments, disclosed herein, can be gained by taking the entire specification, claims, and abstract as a whole.

The current invention disclosure discloses a specially designed educational kit to facilitate an understanding of sexuality and sex in a more effective manner.

It is a primary objective of the current invention to provide a comprehensive education kit to educate young adolescent about sex education.

It is also an objective of the invention to provide an education kit that can conveniently break the silence between parents and their child about sex.

It is yet another objective of the invention to more effectively provide thorough knowledge about the subject matter.

It is further an objective of the invention to provide an educational kit that can be customized according to need.

It is, therefore, an objective of the present invention to provide a new and improved educational kit that has all of the advantages of the prior art educational tools and techniques, and none of the disadvantages.

It is another objective of the present invention to provide a new, improved, and comprehensive educational kit that may be easily and efficiently manufactured and marketed.

It is a further objective of the present invention to provide a new, improved, and comprehensive educational kit that is durable and has reliable construction.

Other objectives of the invention will become apparent to those skilled in the art once the invention has been shown and described. These objectives are not to be construed as limitations of the applicant's invention, but are merely aimed to suggest some of the many benefits that may be realized by the apparatus of the present application with its many embodiments. In addition to the various objectives and advantages of the present invention, described with some degree of specificity above, it should be obvious that additional objectives and advantages of the present invention will become more readily apparent to those persons who are skilled in the relevant art from the following more detailed description of the invention.

### DETAILED DESCRIPTION OF THE INVENTION

The embodiments of the present disclosure, described below, are not intended to be exhaustive or to limit the disclosure to the precise forms disclosed in the following detailed description. Rather, the embodiments are chosen and described so that others skilled in the art may appreciate and understand the principles and practices of the present disclosure.

The following embodiments and the accompanying drawings, which are incorporated into and form part of this disclosure, illustrate embodiments of the invention, and, together with the description, serve to explain the principles of the invention.

The current invention disclosure discloses an educational kit containing a variety of related items necessary to educate adolescents about their sexuality and evident changes that occur to them during puberty.

It has been observed that when a child grows into a teenager, their relationship with parents may change. Parents will find themselves needing to switch from relatively simple parenting to focusing on a new parent adolescent relationship.

Developmentally, teenagers face huge changes physically, mentally, and emotionally. In order to grow up, they must begin to separate from their parents. Although it may not seem so, this process is at least as painful for them as it is for the parents.

These qualitative changes are due to greater cognitive and emotional maturity. As teens become more emotionally mature, their relationships with their peers become more trusting and more emotionally intimate. Cognitive development enables youth to better understand and anticipate the wants, needs, and feelings of their peers. This increased mental and emotional maturity means that adolescents are now better able to offer genuine emotional support and comfort to each other, as well as sensible advice. Thus, the family is no longer the only source of social support.

This is one of the major reasons that limit the conversation between teens and their parents, which creates a troublesome situation for parents to address several issues regarding the changes that are happening in their child's life. Most of the time, parents fail to communicate and educate their teenagers about their sexuality effectively due to this social conditioning.

The educational kit of the current invention contains information about male prevention, samples of female contraceptive, and videos related to childbirth, abortion, and sexually transmitted diseases.

The current invention education kit can be made in accordance with male and female adolescent needs.

For example, a female educational kit box may be comprised of sanitary napkins, tampons, instructions about how to use them, a book about personal hygiene, DVD's related to sexually transmitted diseases, etc.

The educational kit for a male may be comprised of condoms, the method of using condoms, details about maintenance of personal hygiene, information about sexually transmitted diseases, etc.

The kit may be customized by replacement or addition of various types of materials, including DVD's, booklet and props.

Although specific embodiments have been illustrated and described herein, it will be appreciated by those of ordinary skill in the art that any arrangement that is calculated to achieve the same purpose may be substituted for the specific embodiment shown. This application is intended to cover any adaptations or variations of the present invention. Although specific terms are used in this specification, these terms are for convenience of description, are not the limitation of the present invention in any combination.

Although the invention has been explained in relation to its preferred embodiment, it is to be understood that many other possible modifications and variations can be made without departing from the spirit and scope of the invention.

It is to be understood that the above description is intended to be illustrative, and not restrictive.

I claim :

1. An educational kit comprises:
booklets about sexually transmitted diseases;
DVD's about sexually transmitted diseases;
condoms;
sanitary napkins; and
instructions.

2. The educational kit as claimed of claim 1 having information specifically for female adolescents.

3. The educational kit as claimed of claim 1 having information specifically for male adolescents.

4. The educational kit of claim 2 comprises:
a booklet providing explanations of the menstrual cycle;
a DVD complementing said booklet;
a sanitary napkin;
detail information about personal hygiene; and
hygiene accessories.

5. The educational kit for male adolescents of claim 3 may comprise:
a booklet providing explanations of sexually transmitted diseases;
a DVD complementing said booklet;
a condom;
detail information about personal hygiene; and
accessories.

6. The educational kit of claim 1 for training and learning process.
